(19) United States
(12) Patent Application Publication
(10) Pub. No.: US 2002/0102614 A1
(43) Pub. Date: Aug. 1, 2002

(54) Use of ZPR1 as a molecular probe for spinal muscular atrophy

(76) Inventors: Roger J. Davis, Princeton, MA (US); Laxman Gangwani, Worcester, MA (US)

Correspondence Address: J. PETER FASSE, Fish & Richardson P.C., 225 Franklin Street, Boston, MA, 02110-2804, US

(21) Appl. No.: 09/988,915

(22) Filed: Nov. 19, 2001

Related U.S. Application Data
(60) Provisional application No. 60/249,745, filed on Nov. 17, 2000.

(51) Int. Cl. C12Q 1/68; G01N 33/53; C12Q 1/37

(52) U.S. Cl. 435/7.1; 435/23; 435/6

(57) ABSTRACT

The present invention is based on the discovery that ZPR1 and SMN interact in nuclear gems, and that aberrant interaction between them is associated with spinal muscular atrophy. The invention encompasses methods of diagnosing spinal muscular atrophy, methods of assaying for compounds that affect the interaction between ZPR1 and SMN, and antibodies raised against ZPR1 using a B domain epitope.

### CROSS REFERENCE TO RELATED APPLICATION

This application claims priority from U.S. Provisional Application Serial No. 60/249,745 filed on Nov. 17, 2000, which is incorporated herein by reference in its entirety.

### STATEMENT AS TO FEDERALLY SPONSORED RESEARCH

This invention was made with Government support under CA58396 awarded by the National Cancer Institute. The Government has certain rights in the invention.

### TECHNICAL FIELD

This invention relates to the fields of molecular biology, medicine, intracellular transport and signaling, and more particularly to spinal muscular atrophy.

### BACKGROUND

Spinal muscular atrophy (SMA) is a common autosomal recessive disease of early childhood caused by the degeneration of α-motor neurons of the spinal cord anterior horn leading to progressive muscle atrophy, paralysis, respiratory failure, and infant death (Lefebvre et al., 1995, Cell 80:155-165; Jablonka et al., 2000, J. Neurol. 247 Suppl. 1:I37-42). The most severe form of SMA, Werdnig-Hoffman syndrome (SMA type-I), results from deletions or mutations of the survival of motor neuron (SMN1) gene.

Humans have two copies of the SMN gene (SMN1 and SMN2) that are located in a 500 kb inverted repeat on chromosome 5q13 (Lefebvre et al., supra). The human SMN genes differ by a single nucleotide that affects pre-mRNA splicing (Monani et al., 1999, Hum. Mol. Genet. 8:1177-1183; Lorson et al., 1999, Proc. Nat. Acad. Sci. USA 96:6307-6311). The telomeric gene SMN1 primarily generates mRNA transcripts that encode a full-length SMN protein, whereas the centromeric gene SMN2 produces alternatively spliced forms lacking exon 5 and/or exon 7 (Monani et al. supra; Lorson et al. supra). In SMA type-I patients, the SMN1 gene is deleted and the SMN2 gene predominantly produces COOH-terminal truncated proteins lacking sequences derived from exon 7.

Recent studies have provided strong evidence that SMA disease is caused by the expression of SMNΔexon 7 protein (derived from SMN2) in the absence of high levels of the full-length SMN protein. First, cre-mediated deletion of SMN exon 7 in neurons causes SMA disease in mice (Frugier et al., 2000, Hum. Mol. Genet. 9:849-858). Second, transgenic expression of human SMN2 rescues the embryonic lethality of Smn −/−mice and causes SMA disease (Hsieh-Li et al., 2000, Nat. Genet. 24:66-70; Monani et al., 2000, Hum. Mol. Genet. 9:333-339). SMA disease is characterized by low level expression of full-length SMN protein and apoptosis of spinal cord motor neurons.

In normal individuals, the SMN protein is present in the cytoplasm and in small sub-nuclear structures called gems (Liu and Dreyfuss, 1996, EMBO J 15:3555-3565). Cytoplasmic SMN is involved in spliceosomal snRNP biogenesis (Liu et al., 1997, Cell 90:1013-1021; Fischer et al., 1997, Cell 90:1023-1029), while nuclear SMN may play a role in pre-mRNA splicing (Pellizzoni et al, 1998, Cell 95:615-624). The severity of SMA disease negatively correlates with the number of SMN nuclear gems Coover et al, 1997, Hum. Mol. Genet. 6:1205-1214; Lefebvre et al., 1997, Nat. Genet. 16:265-269). Indeed, the absence of SMNΔexon 7 is not observed in the nucleus (Frugier et al., 2000, Hum. Mol. Genet. 9:849-858), thus, the normal function of SMN may require nuclear gems.

ZPR1 (zinc finger protein 1) is a cytoplasmic protein that redistributes from the cytoplasm to the nucleus in response to treatment of mammalian cells with mitogens (Galcheva-Gargova et al., Science 272:1797-1802, U.S. Pat. No. 5,925,566). The function of ZPR1 is unknown, although genetic analysis has demonstrated that it is an essential gene (Galcheva-Gargova et al., 1998, Mol Biol. Cell 9:2963-2971; Gangwani et al, 1998, J. Cell. Biol. 143:1471-1484).

### SUMMARY

The invention is based on the discovery that there is an interaction between ZPR1 (zinc finger protein 1) polypeptide and the SMN (survival of motor neuron 1) protein product, and that ZPR1 polypeptide is required for the localization of the protein product of the SMN gene in nuclear gems. Alterations in ZPR1 polypeptide or ZPR1 polypeptide metabolism may contribute to the pathogenesis of spinal muscular atrophy (SMA). It was also discovered that the B domain of ZPR1 is useful for making antibodies.

The invention provides a method of diagnosing spinal muscular atrophy or a predisposition (e.g., genetic predisposition) to spinal muscular atrophy in a mammal (e.g., human). The method includes the steps of obtaining a biological sample from the mammal, detecting the amount of ZPR1 RNA or ZPR1 polypeptide present, and detecting the level of ZPR1 polypeptide activity per unit protein, or detecting ZPR1 polypeptide localization, relative to a control. A difference in the amount of ZPR1 RNA or ZPR1 polypeptide, ZPR1 polypeptide activity, or ZPR1 localization in the sample compared to the control indicates that the mammal has spinal muscular atrophy or has a predisposition to spinal muscular atrophy. In some embodiments of the invention, the amount of SPR1 RNA or ZPR1 polypeptide, ZPR1 protein activity, or the level of ZPR1 polypeptide interaction with SMN polypeptide is decreased in the sample compared to the control. In another embodiment of the invention, the ZPR1 polypeptide localization to nuclear gems is reduced in the sample compared to the control.

The invention also includes a method of diagnosing spinal muscular atrophy or a predisposition to spinal muscular atrophy in a mammal (e.g., human), in which the method contains the steps of obtaining a biological sample from the mammal, and detecting at least one mutation in both alleles of the ZPR1 gene. The genes may contain the same mutation or different mutations. Mutations causing spinal muscular atrophy or a predisposition to spinal muscular atrophy include mutations in the NH₂-terminal domain that cause a decrease or lack of expression of ZPR1. The mutation can be in the ZPR1 B domain. In general, a control ZPR1 sequence encodes the amino acid sequence of SEQ ID NO:1.

The invention also provides a method of diagnosing spinal muscular atrophy, a predisposition to spinal muscular atrophy, or the spinal muscular atrophy carrier status of a mammal (e.g., human), in which the method includes obtaining a biological sample from the mammal, and detecting a mutation in a ZPR1 gene. In some embodiments of the invention, the mutation in the ZPR1 B domain. In general, the ZPR1 genes from an unaffected individual encode the sequence of SEQ ID NO:1.

Another embodiment of the invention is an isolated ZPR1 B domain polypeptide having the amino acid sequence of SEQ ID NO:2. The isolated polypeptide can be used to make an antibody against the ZPR1 B domain.

The invention also provides a method of making an antibody. The method includes immunizing a mammal with an amount of ZPR1 B domain (residues 298-459 of human ZPR1, SEQ ID NO:2) that is effective to cause an immunologic response to the ZPR1 B domain. The antibody can be a polyclonal antibody or a monoclonal antibody. The invention includes an antibody made by the foregoing method.

The invention also provides an antibody preparation that contains antibodies that specifically bind to (i.e., are exclusively directed against) the ZPR1 B domain. Such antibodies can be made by immunizing a mammal with a ZPR1 B domain. The antibodies can be monoclonal antibodies. The invention also includes an antibody made by immunizing a mammal with a ZPR1 B domain fused to one or more non-ZPR1 peptides. In this method the non-ZPR1 peptide can be a FLAG peptide. In some embodiments of the invention, the antibody specifically binds a ZPR1 B domain. By “specifically binds” is meant a molecule that binds to a particular entity, e.g., a ZPR1 B domain polypeptide, but which does not substantially recognize or bind to other molecules in a sample, e.g., a biological sample, which includes the particular entity, e.g., a ZPR1 B domain polypeptide.

The invention also provides a method of determining whether a compound alters the localization of ZPR1 polypeptide. The method includes the steps of providing a test cell that expresses a ZPR1 polypeptide, contacting the test cell with a candidate compound, and detecting the localization of ZPR1 polypeptide compared to a control cell not contacted with the candidate compound. The compound can affect the localization of ZPR1 in nuclear gems. The test cell can be a cultured cell. In some embodiments of the invention, the compound increases the amount of localization. In other embodiments, the compound decreases the amount of localization. In some embodiments of the invention, the compound alters the amount of co-localization of ZPR1 with SMN.

The invention also provides a method of identifying a compound that modulates the interaction of a ZPR1 polypeptide and SMN polypeptide. The method includes providing a test cell that expresses a ZPR1 polypeptide and an SMN polypeptide, contacting the test cell with a candidate compound, and detecting the interaction of the ZPR1 polypeptide and the SMN polypeptide compared to a control cell not contacted with the candidate compound. In some embodiments of the invention, the compound increases the amount of interaction. In other embodiments of the invention, the compound decreases the amount of interaction. The interaction can be, e.g., specific binding or co-localization.

In some embodiments of the invention, ZPR1 nucleic acid sequence and polypeptide sequence are from a human (e.g., Genbank accession no. AF019767; FIG. 1). In other embodiments, the ZPR1 nucleic acid sequence and polypeptide sequence (including the B domain) are from Schizosaccharomyces pombe (Genbank accession nos. Z97208 and AF019768), Saccharomyces cerevisiae (Genbank accession no. AF019769), Mus musculus (Genbank accession no. U41287), or other ZPR1 sequences (see also, U.S. Pat. No. 5,925,566).

A “polypeptide” means a chain of amino acids regardless of length or post-translational modifications. As used herein, the term “ZPR1” means a ZPR1 polypeptide. As used herein, the term “SMN” means an SMN polypeptide.

A control biological sample is, e.g., from an individual that does not have SMA, does not harbor a gene that predisposes them to SMA, and is not a carrier of a gene that predisposes an individual to SMA (control). A predetermined reference value can also be used in methods of determining whether an individual has SMA, is predisposed to SMA, or is a carrier. Such reference values may be determined relative to controls (e.g., the amount of a ZPR1 RNA or ZPR1 polypeptide present in individuals that do not have SMA and are not carriers of a gene that predisposes to SMA), in which case a test sample that is different from the reference would indicate that the individual has SMA, is predisposed to SMA, or has carrier status. A reference value can also reflect amounts in affected individuals (e.g., the amount of a ZPR1 RNA or ZPR1 polypeptide present in an individual that has SMA, is predisposed to SMA, or has carrier status). In this case, a test sample that is similar to (e.g., equal to or less than) the reference would indicate that the individual has SMA, is predisposed to SMA

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference. In addition, the materials, methods, and examples are illustrative only and not intended to be limiting.

Other features and advantages of the invention will be apparent from the detailed description, and from the claims.

### DESCRIPTION OF DRAWINGS

FIG. 1A is a schematic drawing of ZPR1 that illustrates the two zinc fingers (ZnF1 and ZnF2) and the putative A and B domains. The top panel represents the complete ZPR1 protein and the bottom panel represents the structure of a mutant ZPR1 protein that lacks the B domain (residues 298-459).

FIG. 1B is a schematic drawing of the primary structure of SMN, illustrating the full-length (top panel) and alternatively spliced isoform, Δexon7 (bottom panel).

FIG. 2 depicts the amino acid sequence of human ZPR1 (SEQ ID NO:1).

FIG. 3 depicts the amino acid sequence of the human ZPR1 B domain (SEQ ID NO:2).

FIG. 4 depicts the amino acid sequence of the human ZPR1 NH₂-terminal domain (SEQ ID NO:6).

FIG. 5 depicts the amino acid sequence of the human ZPR1 COOH-terminal domain (SEQ ID NO:7).

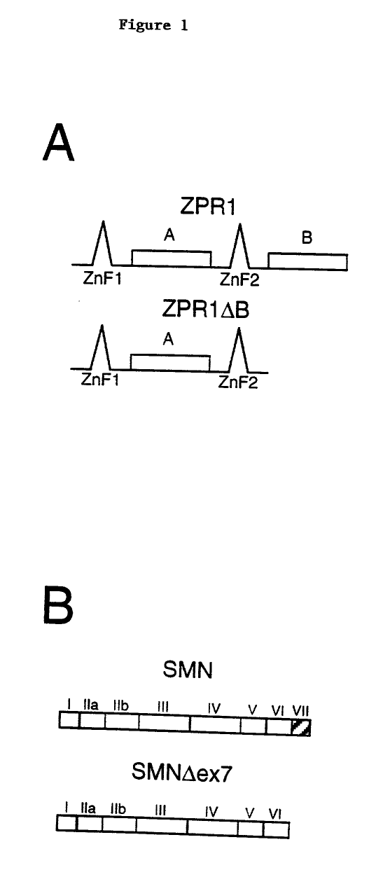

### DETAILED DESCRIPTION

ZPR1 B Domain Epitopes and Antibodies

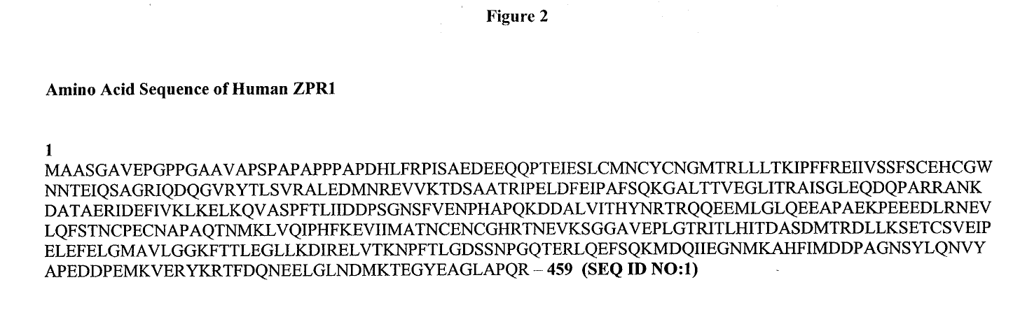

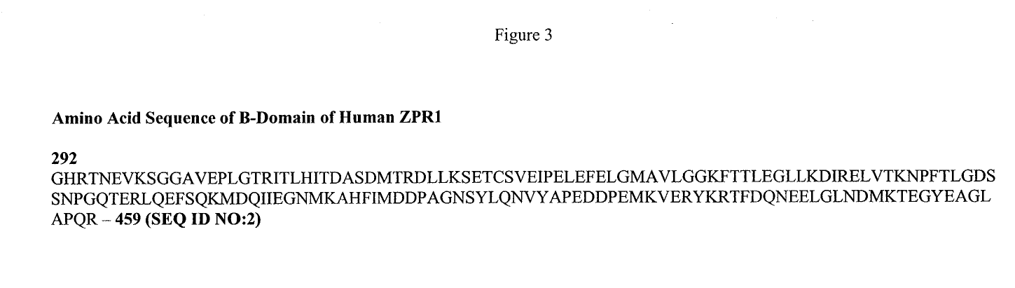

Useful antibodies are those that specifically bind a ZPR1, including those that bind a ZPR1 B domain. A ZPR1 B domain polypeptide fragment can be used as an immunogen to raise antibodies against ZPR1 protein (FIG. 2, SEQ ID NO:1) and B domain epitopes. As used herein, “B domain” means amino acids 298-549 of human ZPR1 (SEQ ID NO:2). As used herein, “B domain epitope” means an epitope of the B domain of a ZPR1 protein. Antibodies that specifically bind the ZPR1 B domain are included in the invention. Also included are methods of making and using such antibodies, e.g. for diagnosis of spinal muscular atrophy.

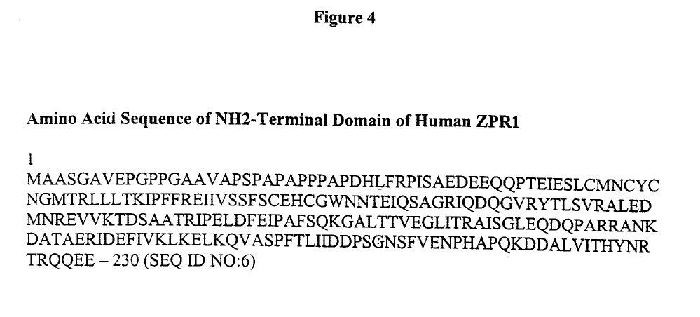

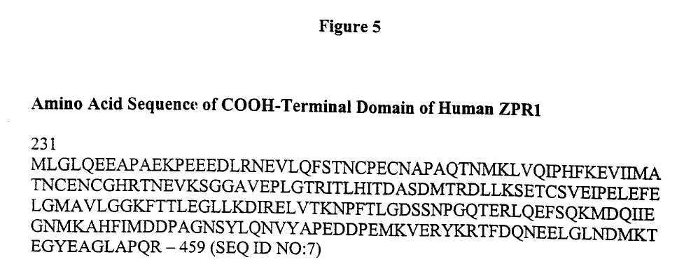

A ZPR1 NH₂-terminal domain polypeptide fragment or an antigenic fragment thereof can be used as an immunogen to raise antibodies against ZPR1 protein and the NH₂-terminal domain. The NH₂-terminal domain of human ZPR1 is pictured in FIG. 4 (SEQ ID NO:6), and comprises amino acids 1-459 of human ZPR1.

As used herein, “isolated” or “purified” ZPR1 polypeptide or ZPR1 B domain means a ZPR1 polypeptide or B domain that is substantially free of cellular material or other contaminating proteins from the cell or tissue source in which the ZPR1 polypeptide naturally occurs. As used herein, “substantially free of cellular material” means separated from cellular components of the cells from which a protein is isolated or recombinantly produced. Thus, ZPR1 B domain that is substantially free of cellular material includes preparations of ZPR1 B domain having less than about 30%, 20%, 10%, or 5% (by dry weight) of non-ZPR1 protein. Such preparations are useful for e.g., raising antibodies against ZPR1 protein or a ZPR1 B domain.

A B domain epitope of a ZPR1 polypeptide can be a polypeptide which is, for example, 10, 25, 50, 72, 100, 125, 150, 160, or 168 amino acids in length.

A useful ZPR1 B domain is one which includes an amino acid sequence at least about 45%, preferably 55%, 65%, 75%, 85%, 95%, or 99% identical to the amino acid sequence of SEQ ID NO:2. This includes the ZPR1 B domains of yeast and mouse ZPR1 sequences referred to herein.

The determination of percent identity between two amino acid sequences is accomplished using the BLAST 2.0 program, which is available to the public at http://www.ncbi.nlm.nih.gov/BLAST. Sequence comparison is performed using an ungapped alignment and using the default parameters (Blossom 62 matrix, gap existence cost of 11, per residue gapped cost of 1, and a lambda ratio of 0.85). The mathematical algorithm used in BLAST programs is described in Altschul et al., 1997, Nucleic Acids Res. 25:3389-3402.

In one embodiment, a useful fusion protein is a B domain-1-immunoglobulin fusion protein in which all or part of B domain sequence is fused to sequences derived from a member of the immunoglobulin protein family. The B domain-1-immunoglobulin fusion proteins can be used as immunogens to produce anti-ZPR1 antibodies in an animal, to purify ZPR1 ligands, and in screening assays to identify molecules which inhibit the interaction of ZPR1 polypeptide or a B domain with a ZPR1 ligand (e.g., SMN).

A ZPR1 chimeric or fusion protein can be produced by conventional recombinant DNA techniques. For example, DNA fragments coding for the different polypeptide sequences are ligated together in-frame in accordance with conventional techniques. This can be done using blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, e.g., Current Protocols in Molecular Biology, Ausubel et al. eds., John Wiley & Sons: 1992). Moreover, many expression vectors are commercially available that already encode a fusion moiety (e.g., a GST polypeptide). A ZPR1-encoding nucleic acid can be cloned into such an expression vector such that the sequence encoding the fusion moiety is linked in-frame to the sequence encoding the B domain.

An isolated B domain epitope can be used as an immunogen to generate antibodies that bind ZPR1 polypeptide or B domain using standard techniques for polyclonal and monoclonal antibody preparation. The invention specifically encompasses immunogens that include an antigenic peptide that includes all or part of a ZPR1 B domain. The antigenic peptide of B domain comprises at least 8 (preferably 10, 15, 20, or 30) amino acid residues of the amino acid sequence shown in SEQ ID NO:2 such that an antibody raised against the peptide forms a specific immune complex with ZPR1 polypeptide or B domain.

A B domain epitope immunogen is used to prepare antibodies by immunizing a suitable animal (e.g., rabbit, goat, mouse, or other mammal) with the immunogen. An appropriate immunogenic preparation can contain, for example, recombinantly expressed B domain or a chemically synthesized B domain polypeptide. The preparation can further include an adjuvant, such as Freund's complete or incomplete adjuvant, or similar immunostimulatory agent. Immunization with an immunogenic B domain epitope preparation induces a polyclonal anti-ZPR1 or B domain antibody response.

The invention includes anti-B domain antibodies. As used herein, “antibody” means immunoglobulin molecules and immunologically active portions of immunoglobulin molecules, i.e., molecules that contain an antigen binding site which specifically binds an antigen, such as a ZPR1 polypeptide.

Examples of immunologically active portions of immunoglobulin molecules include F(ab) and F(ab′)2 fragments which can be generated by treating the antibody with an enzyme such as pepsin. The invention provides polyclonal and monoclonal antibodies that bind ZPR1 or B domain. The term “monoclonal antibody” or “monoclonal antibody composition”, as used herein, refers to a population of antibody molecules that contain only one species of an antigen binding site capable of immunoreacting with a particular epitope of ZPR1. A monoclonal antibody composition thus typically displays a single binding affinity for a particular B domain epitope with which it immunoreacts.

Polyclonal anti-B domain epitope antibodies can be prepared as described above by immunizing a suitable subject with a B domain epitope immunogen. The anti-B domain epitope antibody titer in the immunized subject can be monitored over time by standard techniques, such as with an enzyme linked immunosorbent assay (ELISA) using immobilized ZPR1 protein or B domain. If desired, the antibody molecules directed against ZPR1 protein or B domain can be isolated from the mammal (e.g., from the blood) and further purified by well-known techniques, such as protein A chromatography to obtain the IgG fraction. At an appropriate time after immunization, e.g., when the anti-ZPR1 antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing various antibodies monoclonal antibody hybridomas is well known (see generally Current Protocols in Immunology (1994) Coligan et al. (eds.) John Wiley & Sons, Inc., New York, N.Y.).

Any of the many well known protocols used for fusing lymphocytes and immortalized cell lines can be applied for the purpose of generating a B domain epitope monoclonal antibody (see, e.g., Current Protocols in Immunology, supra; Galfre et al., 1977, Nature 266:55052; R. H. Kenneth, in Monoclonal Antibodies: A New Dimension In Biological Analyses, Plenum Publishing Corp., New York, N.Y., 1980; and Lerner, 1981, Yale J. Biol. Med., 54:387-402). Moreover, the ordinarily skilled worker will appreciate that there are many variations of such methods that also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line, e.g., a myeloma cell line that is sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind a B domain epitope, e.g., using a conventional ELISA assay.

Alternative to preparing monoclonal antibody-secreting hybridomas, a monoclonal anti-ZPR1 or B domain antibody can be identified and isolated by screening a recombinant combinatorial immunoglobulin library (e.g., an antibody phage display library) with ZPR1 or B domain to thereby isolate immunoglobulin library members that bind ZPR1 or a B domain epitope. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734.

Additionally, recombinant anti-B domain antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Canc. Res. 47:999-1005; Wood et al., 1985, Nature 314:446-449; and Shaw et al., 1988, J. Natl. Cancer Inst. 80:1553-1559; Morrison, 1985, Science 229:1202-1207; Oi et al., 1986, Bio/Techniques 4:214; U.S. Pat. N0. 5,225,539; Jones et al., 1986, Nature 321:552-525; Verhoeyan et al., 1988, Science 239:1534; and Beidler et al., 1988, J. Immunol. 141:4053-4060.

An anti-ZPR1 or B domain antibody (e.g., monoclonal antibody) is useful for isolating a ZPR1 protein or fragment by standard techniques, such as affinity chromatography or immunoprecipitation. For example, an anti-ZPR1 or B domain antibody can be used to facilitate the purification of natural ZPR1 from cells and of recombinantly produced ZPR1 or B domain expressed in host cells. Such antibodies are also useful for detecting a ZPR1 or a B domain protein (e.g., in a cellular lysate or cell supernatant) in order to evaluate the abundance, pattern of expression, and localization of the ZPR1 protein or B domain. Anti-ZPR1 or B domain antibodies can be used diagnostically to monitor protein levels in tissue as part of a clinical testing procedure, e.g., to, for example, determine the efficacy of a given treatment regimen for spinal muscular atrophy. Antibody detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H. Also useful for these methods are antibodies that specifically bind the NH₂-terminal domain of a ZPR1 (e.g., FIG. 4; SEQ ID NO:6).

An agent for detecting ZPR1 polypeptide (e.g., the NH₂-terminal domain) or B domain (e.g., for use in diagnosing spinal muscular atrophy) can be an antibody capable of binding to a ZPR1 polypeptide or B domain, preferably an antibody with a detectable label. Such antibodies can be polyclonal, or more preferably, monoclonal, and can be generated using the methods described herein. An intact antibody, or a fragment thereof (e.g., Fab or F(ab′)2) can be used. The term “labeled”, with regard to the probe or antibody, means direct labeling of the probe or antibody by coupling a detectable atom or moiety to the probe or antibody, as well as indirect labeling of the probe or antibody by reactivity with another reagent. Examples of indirect labeling include detection of a primary antibody using a fluorescently labeled secondary antibody and end-labeling of a DNA probe with biotin such that it can be detected with fluorescently labeled streptavidin. The term “biological sample” means tissues, cells, biological fluids, and stool samples isolated from a subject, as well as tissues, cells and fluids present within a subject. In some aspects of the invention, it is useful to detect ZPR1 polypeptide or B domain mRNA, protein, or genomic DNA in a biological sample in vitro as well as in vivo. For example, in vitro techniques for detection of ZPR1 polypeptide or B domain mRNA include Northern hybridizations and in situ hybridizations. In vitro techniques for detection of ZPR1 polypeptide or B domain include enzyme linked immunosorbent assays (ELISAs), Western blots, immunoprecipitations and immunofluorescence. Furthermore, in vivo techniques for detection of ZPR1 polypeptide or B domain include introducing into a subject a labeled anti-ZPR1 or B domain antibody. For example, the antibody can be labeled with a radioactive marker whose presence and location in a subject can be detected by standard imaging techniques.

In one embodiment, the biological sample contains protein molecules from the test subject (e.g., a human). Alternatively, the biological sample can contain mRNA molecules from the test subject or genomic DNA molecules from the test subject. A biological sample can be a peripheral blood leukocyte sample isolated by conventional means from a subject.

The invention also encompasses kits for detecting the presence of ZPR1 polypeptide in a biological sample (a test sample) to determine if a subject is suffering from or is at increased risk of developing spinal muscular atrophy or other disorder associated with aberrant expression or localization of ZPR1. For example, the kit can comprise a labeled compound or agent capable of detecting ZPR1 polypeptide or B domain or a ZPR1 mRNA in a biological sample and means for determining the amount of ZPR1 polypeptide or B domain in the sample (e.g., an anti-ZPR1 or B domain antibody or an oligonucleotide probe which binds to DNA encoding full-length ZPR1 polypeptide (e.g., Genbank Accession No. AF019767), a fragment thereof, or B domain. Kits may also include instructions for observing that the tested subject is suffering from or has a genetic predisposition for developing spinal muscular atrophy that is associated with aberrant expression of ZPR1 polypeptide or B domain if the amount of ZPR1 polypeptide or B domain or a ZPR1 mRNA is above or below the normal level in control subjects.

For antibody-based kits, the kit may comprise, for example: (1) a first antibody (e.g., attached to a solid support) which binds to a ZPR1 polypeptide or B domain; and, optionally, (2) a second, different antibody which binds to SMN polypeptide or the first antibody and is conjugated to a detectable agent. For oligonucleotide-based kits, the kit may comprise, for example: (1) a oligonucleotide, e.g., a detectably labeled oligonucleotide, which hybridizes to a ZPR1 or B domain nucleic acid sequence or (2) a pair of primers useful for amplifying a ZPR1 or B domain nucleic acid molecule. The kit may also contain written instructions.

The kit may also comprise, e.g., a buffering agent, a preservative, or a protein stabilizing agent. The kit may also comprise components necessary for detecting the detectable agent (e.g., an enzyme or a substrate). The kit may also contain a control sample or a series of control samples that can be assayed and compared to the test sample contained. Each component of the kit is usually enclosed within an individual container and all of the various containers are within a single package along with instructions for observing whether the tested subject is suffering from or is at risk of developing spinal muscular atrophy.

Detection of ZPR1/SMN Interactions and Localization

ZPR1/SMN1 interactions can be assessed using methods known in the art such as transfection assays, binding assays and immunohistochemical detection of co-localization Examples of such methods are provided in Examples 1,2,3, and 5 (infra). The methods include indirect measures such as the processing of pre-mRNA. Direct methods of measuring the interaction between ZPR1 and SMN can be used. The methods include localizing both ZPR1 polypeptide and SMN polypeptide within a single cell and evaluating the patterns of localization (e.g., as in Examples 1 and 3).

Prognostic Assays

The methods described herein can be utilized as diagnostic or prognostic assays to identify a subject (e.g., a non-human mammal such as a mouse, or a human) having spinal muscular atrophy or having a genetic predisposition to developing spinal muscular atrophy or a disease or disorder associated with aberrant ZPR1 polypeptide expression or activity. The polypeptide can be aberrant in a B domain or other region of ZPR1. For example, the assays described herein, including the diagnostic assays described herein, can be utilized to identify a subject having spinal muscular atrophy or who has a genetic predisposition for developing spinal muscular atrophy by assaying ZPR1 polypeptide or B domain protein or nucleic acid expression, ZPR1 protein activity, or ZPR1 localization. Thus, the present invention provides a method in which a test sample is obtained from a subject and ZPR1 polypeptide or B domain protein or nucleic acid (e.g., mRNA, genomic DNA) is detected, wherein the abnormal activity (e.g., localization) or expression of ZPR1 or B domain protein or nucleic acid is diagnostic for a subject having spinal muscular atrophy or who has a genetic predisposition for developing spinal muscular atrophy. Assays for activity or expression of the NH₂ terminal domain of ZPR1 are useful. Deletion or mutation of the NH₂-terminal domain can result in a loss of expression of ZPR1. To detect lack of ZPR1 nucleci acid expression, any nucleic acid sequence that specifically detects ZPR1 expression can be used. Similarly, the lack of ZPR1 polypeptide can be detected using any ZPR1 antibody, including those detecting the B-domain, the NH₂-terminal domain, or the COOH-terminal domain (e.g., SEQ ID NOS:2, 6, and 7, respectively). The COOH-terminal domain of human ZPR1 comprises amino acids 231-459 of human ZPR1.

As used herein, a “test sample” means a biological sample obtained from a subject of interest (e.g., a non-human mammal or a human). For example, a test sample can be a biological fluid (e.g., serum), cell sample, tissue (e.g., muscle), or stool sample. Samples may be analyzed using various in vitro techniques, including techniques directed to analysis of DNA, RNA, or protein in the sample (e.g., see Machiels et al., 2000, BioTechniques 28:286-290).

The prognostic assays described herein can be used to determine whether a subject is a candidate for administration of a particular agent (e.g., an agonist, antagonist, peptidomimetic, protein, peptide, nucleic acid, small molecule, or other drug candidate) to treat SMA. Such methods can be used to determine whether a subject can be effectively treated with a specific agent or class of agents (e.g., agents that alter ZPR1 polypeptide activity). For example, an individual having SMA associated with under expression of ZPR1 may benefit from treatment with an agent that increases ZPR1 expression or half-life.

Diagnosis or Prediction of a Predisposition to Spinal Muscular Atrophy

The invention can be used to diagnose SMA by detecting genetic lesions or mutations in a ZPR1 gene that affect ZPR1 expression, activity, or localization. An individual with a lesioned ZPR1 gene may have spinal muscular atrophy or a genetic predisposition to spinal muscular atrophy. In some embodiments, the methods include detecting, in a sample of cells from the subject, the presence or absence of a genetic lesion in a ZPR1 gene characterized by an alteration in the ability of the protein product of the gene to interact with SMN. For example, such genetic lesions can be detected by ascertaining the existence of at least one of: 1) a deletion of one or more nucleotides from a portion of a ZPR1 polypeptide that is involved in the interaction of ZPR1 with SMN (e.g., the NH₂-terminal domain of a ZPR1); 2) an addition of one or more nucleotides to a portion of a ZPR1 gene that encodes a region that is involved in the interaction of ZPR1 with SMN; 3) a substitution of one or more nucleotides of a ZPR1 gene that encodes a region that is involved in the interaction of ZPR1 with SMN (e.g. in the NH₂-terminal domain of ZPR); 4) a chromosomal rearrangement of a ZPR1 gene; 5) an alteration in the level of a messenger RNA transcript of a ZPR1 gene; 6) aberrant modification of a ZPR1 gene, such as of the methylation pattern of the genomic DNA; 7) the presence of a non-wild type splicing pattern of a messenger RNA transcript of a ZPR1 gene (e.g., caused by a mutation in a splice donor or splice acceptor site); 8) a non-wild type level of a ZPR1-protein; 9) allelic loss of a ZPR1 gene; and 10) inappropriate post-translational modification of a ZPR1-protein. As described herein, there are a large number of assay techniques known in the art that can be used for detecting lesions in a ZPR1 gene.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al., 1988, Science 241:1077-1080; and Nakazawa et al., 1994, Proc. Natl. Acad. Sci. USA 91:360-364), the latter of which can be particularly useful for detecting point mutations in a ZPR1 gene (see, e.g., Abravaya et al., 1995, Nucleic Acids Res. 23:675-682). This method can include diagnosing SMA or a genetic predisposition to SMA with the steps of collecting a sample of cells from a patient, isolating nucleic acid (e.g., genomic, mRNA, or both) from the cells of the sample, contacting the nucleic acid sample with one or more primers which specifically hybridize to a ZPR1 gene under conditions such that hybridization and amplification of the ZPR1-gene (if present) occurs, and detecting the presence or absence of an amplification product, or detecting the size of the amplification product and comparing the length to a control sample. It may be desirable to use PCR and/or LCR as a preliminary amplification step in conjunction with any of the techniques used for detecting mutations described herein.

Diagnosis of SMA can also be performed by analyzing mutations in a ZPR1 gene from a sample cell by identifying alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,498,531) can be used to score for the presence of specific mutations by development or loss of a ribozyme cleavage site.

In other embodiments, genetic mutations in a ZPR1 gene that are associated with SMA can be identified by hybridizing a sample and control nucleic acids, e.g., DNA or RNA, to high density arrays containing hundreds or thousands of oligonucleotide probes (Cronin et al., 1996, Human Mutation 7:244-255; Kozal et al., 1996, Nature Medicine 2:753-759). For example, genetic mutations in ZPR1 can be identified in two-dimensional arrays containing light-generated DNA probes as described in Cronin et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

Sequencing reactions known in the art can be used to directly sequence a ZPR1 gene and detect mutations that are associated with SMA. In these methods, the sequence of the sample ZPR1 (e.g., a sample from an individual suspected of having spinal muscular atrophy) is compared with the corresponding sequence from an individual that does not have SMA, does not harbor a gene that predisposes them to SMA, and is not a carrier of a gene that predisposes an individual to SMA (control). Examples of sequencing reactions include those based on techniques developed by Maxam and Gilbert (1977, Proc. Natl. Acad. Sci. USA 74:560) or Sanger (1977, Proc. Natl. Acad. Sci. USA 74:5463). It is also contemplated that any of a variety of automated sequencing procedures can be utilized when performing the diagnostic assays (1995, Bio/Techniques 19:448), including sequencing by mass spectrometry (see, e.g., PCT Publication No. WO 94/16101; Cohen et al., 1996, Adv. Chromatogr. 36:127-162; and Griffin et al., 1993, Appl. Biochem. Biotechnol. 38:147-159).

Other methods for detecting mutations in a ZPR1 gene that are useful for diagnosing SMA include methods in which protection from cleavage agents is used to detect mismatched bases in RNA/RNA or RNA/DNA heteroduplexes (Myers et al., 1985, Science 230:1242). In general, the art technique of “mismatch cleavage” starts by providing heteroduplexes of formed by hybridizing (labeled) RNA or DNA containing the wild-type ZPR1 sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton et al. (1988, Proc. Natl Acad Sci USA 85:4397; Saleeba et al., 1992, Methods Enzymol. 217:286-295. In an embodiment, the control DNA or RNA can be labeled for detection.

In other embodiments, alterations in electrophoretic mobility can be used to identify mutations in ZPR1 genes. For example, single-strand conformation polymorphism (SSCP) may be used to detect differences in electrophoretic mobility between mutant and wild type nucleic acids (Orita et al., 1989, Proc. Natl. Acad. Sci USA: 86:2766, see also Cotton, 1993, Mutat. Res. 285:125-144; and Hayashi, 1992, Genet. Anal. Tech. Appl. 9:73-79). In this method, single-stranded DNA fragments of sample and control ZPR1 nucleic acids are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In an embodiment, the method of detecting a ZPR1 mutation utilizes heteroduplex analysis to separate double-stranded heteroduplex molecules on the basis of changes in electrophoretic mobility (Keen et al., 1991, Trends Genet. 7:5).

In yet another embodiment, a ZPR1 mutation is detected by assaying the movement of mutant or wild-type fragments in polyacrylamide gels containing a gradient of denaturant using denaturing gradient gel electrophoresis (DGGE) (Myers et al., 1985, Nature 313:495). When DGGE is used as the method of analysis, DNA is modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987, Biophys. Chem. 265:12753).

Other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al., 1986, Nature 324:163; Saiki et al., 1989, Proc. Natl. Acad. Sci. USA 86:6230). Such allele-specific oligonucleotides are hybridized to PCR amplified target DNA or a number of different mutations when the oligonucleotides are attached to the hybridizing membrane and hybridized with labeled target DNA.

Allele-specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al., 1989, Nucleic Acids Res. 17:2437-2448) or at the extreme 3′ end of one primer where, under appropriate conditions, mismatch can prevent, or reduce polymerase extension (Prossner, 1993, Tibtech 11:238).

The methods described herein may be performed, for example, by utilizing pre-packaged diagnostic kits comprising at least one probe nucleic acid or antibody reagent (e.g., a B domain antibody) described herein, which may be used, e.g., to diagnose patients exhibiting symptoms or family history of a spinal muscular atrophy.

Furthermore, any cell type or tissue in which a ZPR1 gene is expressed (e.g., muscle) may be utilized in the prognostic assays described herein.

Monitoring of Effects During Clinical Trials

Monitoring the influence of agents (e.g., drugs, compounds) on the interaction of ZPR1 and SMN can be applied not only in basic drug screening, but also in clinical trials. For example, the effectiveness of an agent determined by a screening assay as described herein to alter the interaction of ZPR1 polypeptide and SMN polypeptide, including by assaying the binding or localization of ZPR1, can be monitored in clinical trials of subjects exhibiting decreased ZPR1 or SMN gene expression, protein levels, altered ZPR1 or SMN activity or abnormal localization. In such clinical trials, the localization of ZPR1 polypeptide or the interaction of ZPR1 polypeptide and SMN polypeptide can be used as a “read out” or markers of spinal muscular atrophy.

Thus, to study the effect of agents on SMA, for example, in a clinical trial, cells can be isolated and RNA prepared and analyzed for the levels of expression of ZPR1, and optionally, other genes implicated in the disorder. The levels of gene expression (i.e., a gene expression pattern) can be quantified by Northern blot analysis or RT-PCR, as described herein, by measuring the amount of protein produced, by one of the methods as described herein, by measuring the levels of activity of ZPR1. In this way, the gene expression pattern can serve as a marker, indicative of the physiological response of the cells to the agent. Alternatively, this is accomplished by assaying the interaction of ZPR1 and SMN. Accordingly, the state of the cells may be determined before, and at various points during, treatment of the individual with the agent.

The present invention includes a method for monitoring the effectiveness of treatment with an agent (e.g., an agonist, antagonist, peptidomimetic, protein, peptide, nucleic acid, small molecule, or other drug candidate identified by the screening assays described herein) of an individual having SMA. The method comprises the steps of (i) obtaining a pre-administration sample from a subject prior to administration of the agent; (ii) detecting the level of expression or activity of a ZPR1 protein, mRNA, or genomic DNA, the localization of a ZPR1 protein or the interaction of ZPR1 and SMN in the preadministration sample; (iii) obtaining one or more post-administration samples from the subject; (iv) detecting the level of expression or activity of the ZPR1 protein, mRNA, or genomic DNA, the localization of a ZPR1 protein in the post-administration samples or the interaction of ZPR1 and SMN; (v) comparing the level of expression or activity of the ZPR1 protein, mRNA, or genomic DNA, the localization of a ZPR1 protein or the interaction of ZPR1 and SMN in the pre-administration sample with the post administration sample or samples; and (vi) altering the administration of the agent to the subject accordingly. For example, increased administration of the agent may be desirable to increase the expression or activity of ZPR1 to higher levels than detected or to increase the interaction of ZPR1 and SMN, i.e., to increase the effectiveness of the agent. Alternatively, decreased administration of the agent may be desirable to decrease expression or activity of a ZPR1 protein, or to increase the interaction of ZPR1 and SMN to lower levels than detected, i.e., to decrease the effectiveness of the agent.

Screening Assays

The invention provides a method for identifying modulators, i.e., candidate or test compounds or agents (e.g., polypeptides, peptidomimetics, small inorganic molecules, or small organic molecules (e.g., small non-nucleic acid organic molecules) that modulate the localization of ZPR1 polypeptide. The invention also includes methods of identifying compounds that modulate the interaction of ZPR1 polypeptide and SMN polypeptide.

Compounds that affect the localization of ZPR1 polypeptide or modulate the interaction of ZPR1 polypeptide and SMN polypeptide may be selected from those compounds discovered to bind to ZPR1 polypeptide or SMN polypeptide. Methods of assay for such compounds are known in the art.

In one embodiment, an assay is a cell-based assay in which a cell which expresses a ZPR1 polypeptide or, or a biologically active portion thereof, is contacted with a test compound and the ability of the test compound to alter the localization of ZPR1 polypeptide is determined. Localization of ZPR1 can be accomplished using methods known in the art such as cell fractionation followed by assay for ZPR1 activity, protein expression, or immunoreactivity. Another method that can be used to determine the localization is by histological methods in which ZPR1 polypeptide is localized using an antibody. Histological localization can be performed using light microscopy, electron microscopy, confocal microscopy, or other suitable methods.

In one embodiment, an assay is a cell-free assay comprising contacting a ZPR1 polypeptide or biologically active portion thereof with a test compound and determining the ability of ZPR1 to bind to SMN in the presence of the test compound.

In one embodiment of the invention, an assay is a cell-free assay that comprises contacting a ZPR1 polypeptide or biologically active portion thereof with an SMN polypeptide which binds the ZPR1 polypeptide to form an assay mixture, contacting the assay mixture with a test compound, and determining the ability of the test compound to alter the binding of the ZPR1 and the SMN, wherein determining the ability of the test compound to alter the interaction of ZPR1 and SMN comprises determining the ability of the ZPR1 polypeptide to bind to SMN.

In some embodiments of the invention, it may be desirable to immobilize either the ZPR1 polypeptide or the SMN polypeptide to facilitate separation of complexed from uncomplexed forms of one or both of the proteins, as well as to accommodate automation of the assay. Cell-free assays can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase ZPR1 fusion proteins or glutathione-S-Sepharose SMN fusion proteins can be adsorbed onto glutathione Sepharose beads (Sigma Chemical; St. Louis, Mo.) or glutathione derivatized microtitre plates, which are then combined with SMN polypeptide or ZPR1 polypeptide and the test compound. The mixture is incubated under conditions conducive to complex formation (e.g., at physiological conditions for salt and pH). Following incubation, the beads or microtitre plate wells are washed to remove any unbound components and complex formation is measured either directly or indirectly, for example, using immunohistochemical detection. Alternatively, the complexes can be dissociated from the matrix, and the level of binding or activity of the polypeptide can be determined using conventional techniques.

Other techniques for immobilizing proteins on matrices can also be used in the screening assays of the invention. For example, either a ZPR1 polypeptide or an SMN polypeptide can be immobilized utilizing conjugation of biotin and streptavidin. Biotinylated ZPR1 polypeptide or SMN polypeptide can be prepared from biotin-NHS (N-hydroxy-succinimide) using techniques well known in the art (e.g., biotinylation kit, Pierce Chemicals; Rockford, Ill.), and immobilized in the wells of streptavidin-coated 96 well plates (Pierce Chemical). Alternatively, antibodies reactive with ZPR1 or SMN but which do not interfere with binding of ZPR1 to SMN can be derivatized to the wells of the plate, and unbound SMN polypeptide or ZPR1 polypeptide trapped in the wells by antibody conjugation. Methods for detecting such complexes, in addition to those described above for the GST-immobilized complexes, include immunodetection of complexes using antibodies reactive with the ZPR1 polypeptide or the SMN polypeptide.

Other suitable assays that detect compounds that alter the interaction of ZPR1 polypeptide and SMN polypeptide can be used, e.g., a two-hybrid screening method such as that described by Vidal (U.S. Pat. No. 5,965,368).

Treatment of Spinal Muscular Atrophy

Pharmaceutical Compositions

Compounds that affect ZPR1 RNA expression or activity, protein expression or activity, protein localization or the interaction of ZPR1 polypeptide and SMN polypeptide (also referred to herein as “active compounds”) can be incorporated into pharmaceutical compositions suitable for administration to individuals having SMA. Such compositions typically comprise the active compound and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The invention includes methods for preparing pharmaceutical compositions for modulating the expression or activity of a ZPR1 polypeptide or nucleic acid, altering the localization of ZPR1 polypeptide, or altering the interaction of ZPR1 polypeptide and SMN polypeptide. Such methods comprise formulating a pharmaceutically acceptable carrier with an agent that modulates expression or activity of a ZPR1 polypeptide or nucleic acid, alters the localization of ZPR1 polypeptide, or alters the interaction of ZPR1 polypeptide and SMN polypeptide. Such compositions can further include additional active agents. Thus, the invention further includes methods for preparing a pharmaceutical composition by formulating a pharmaceutically acceptable carrier with an agent that modulates expression or activity of a ZPR1 polypeptide or nucleic acid, alters the localization of ZPR1 polypeptide, or alters the interaction of ZPR1 polypeptide and SMN polypeptide and one or more additional active compounds.

The agent that modulates expression or activity may, for example, be a small molecule. For example, such small molecules include peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight les than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the ZPR1 nucleic acid or polypeptide. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to a subject (e.g., a non-human mammal or a human) in order to modulate expression or activity of a ZPR1 polypeptide or nucleic acid, alter ZPR1 polypeptide localization, or alter the interaction of ZPR1 polypeptide and SMN polypeptide, a physician, veterinarian, or researcher may, for example, prescribe a relatively low dose at first, subsequently increasing the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

A pharmaceutical composition of the invention is formulated to be compatible with its intended route of administration. Examples of routes of administration include oral or parenteral, e.g., intravenous, intradermal, subcutaneous, inhalation, transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a compound that affects the localization of ZPR1 polypeptide or interaction between ZPR1 polypeptide and SMN polypeptide) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

As used herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of protein, polypeptide, or antibody can include a single treatment or, preferably, can include a series of treatments.

For antibodies, the preferred dosage is 0.1 mg/kg to 100 mg/kg of body weight (generally 10 mg/kg to 20 mg/kg). Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997, J. Acquired Immune Deficiency Syndromes and Human Retrovirology 14:193).

Nucleic acid molecules can be inserted into vectors and used as gene therapy vectors. Gene therapy vectors can be delivered to an individual having SMA or genetically predisposed to SMA by, for example, intravenous injection, local administration (U.S. Pat. No. 5,328,470) or by stereotactic injection (see, e.g., Chen et al., 1994, Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The gene therapy vectors can be either viral or non-viral. Examples of plasmid-based, non-viral vectors are discussed in Huang et al. (1999) Nonviral Vectors for Gene Therapy (supra). A modified plasmid is one example of a non-viral gene delivery system. Peptides, proteins (including antibodies), and oligonucleotides may be stably conjugated to plasmid DNA by methods that do not interfere with the transcriptional activity of the plasmid (Zelphati et al. (2000) BioTechniques 28:304-315). The attachment of proteins and/or oligonucleotides may influence the delivery and trafficking of the plasmid and thus render it a more effective pharmaceutical composition.

The pharmaceutical compositions can be included in a container, pack, or dispenser together with instructions for administration.

Gene Therapy Methods

The invention includes a method of treating a non-human mammal or a human with spinal muscular atrophy comprising introducing into the non-human mammal or human an expression vector encoding a ZPR1 polypeptide such that an amount of ZPR1 polypeptide effective to alleviate the symptoms of spinal muscular atrophy is expressed in the non-human mammal or in the human.

A viral delivery system for a ZPR1 nucleic acid molecule can be used for such gene therapy, e.g., adenoviral or retroviral gene delivery systems, to treat spinal muscular atrophy.

A non-viral delivery system for a ZPR1 nucleic acid molecule can also be used for gene therapy to treat spinal muscular atrophy. Thus, another aspect of the invention pertains to such uses of non-viral gene delivery systems, such as plasmid-based gene delivery systems. Non-viral gene delivery systems are described in detail by Huang et al. (1999, Nonviral Vectors for Gene Therapy, Academic Press, San Diego, Calif.). Nonviral vectors have several potential advantages over their viral counterparts, including: reduced immunogenicity; low acute toxicity; simplicity; and ease of large scale production. Nonviral vectors can be delivered as naked DNA, by bioballistic bombardment, and in various complexes, including liposome/DNA complexes (lipoplexes), polymer/DNA complexes (polyplexes), and liposome/polymer/DNA complexes (lipopolyplexes). Nonviral vectors may be administered by various routes, e.g., intravenous injection, peritoneal injection, intramuscular injection, subcutaneous injection, intratracheal injection, and aerosolization.

Naked DNA (i.e. free from association with, e.g., transfection-facilitating proteins, viral particles, liposomal formulations, charged lipids and calcium phosphate precipitating), can be expressed at its injection site or at a remote site and so is also useful for delivering a ZPR1 nucleic acid molecule to a subject with spinal muscular atrophy. For example, naked DNA can be injected directly into skeletal muscle, liver, heart muscle, and tumor tissue. For systemic administration, plasmid DNA may need to be protected from degradation by endonucleases during delivery from the site of administration to the site of gene expression.

Bioballistic bombardment, also known as gene gun, allows for the penetration of target cells in vitro, ex vivo, or in vivo and can be used to deliver a ZPR1 nucleic acid molecule to treat a subject with spinal muscular atrophy. In this technique, DNA-coated gold particles are accelerated to a high velocity by an electric arc generated by a high voltage discharge. The method is effective for a variety of organ types, including skin, liver, muscle, spleen, and pancreas. The gene gun transfer method is not dependent upon specific cell surface receptors, cell cycle status, or the size of the DNA vector. Useful gene gun devices include the Accell® (PowderJect Vaccines, Inc.) and the Helios™ (Bio-Rad). These devices create a compressed shock wave of helium gas, accelerating DNA-coated gold (or tungsten) particles to high speed, whereby the particles have sufficient momentum to penetrate a target tissue.

Lipoplexes are typically made up of three components: a cationic lipid, a neutral colipid, and plasmid DNA that encodes one or more genes of interest. Commonly used cationic lipids include DOTMA, DMRIE, DC-chol, DOTAP, DMRIE, DDAB, DODAB/C, DOGS, DOSPA, SAINT-n, DOSPER, DPPES, DORIE, GAP-DLRIE, and DOTIM. Dioleoyl (DO) and dimyristoyl (DM) chains are thought to be especially effective for gene delivery. Thus, lipoplexes are useful for treatment of spinal muscular atrophy as a means of delivering a ZPR1 nucleic acid. Cationic lipids are typically composed of a positively charged headgroup, a hydrophobic lipid anchor, and a linker that connects the headgroup and anchor. Cationic lipids used in lipoplexes can be divided into two broad classes: those that use cholesterol as the lipid anchor and those that use diacyl chains of varying lengths and extent of saturation. The number of protonatable amines on the headgroup may affect transfection activity, with multivalent headgroups being generally more active than monovalent headgroups. The linker can be made of a variety of chemical structures, e.g., ether, amide, carbamate, amine, urea, ester, and peptide bonds. Neutral colipids of lipoplexes commonly include DOPE, DOPC, and cholesterol. Generally, DOPE is used as the neutral colipid with cationic lipids that are based on cholesterol (e.g., DC-chol, GL-67) and cholesterol is used as the neutral colipid with cationic lipids that harbor diacyl chains as the hydrophobic anchor (e.g., DOTAP, DOTIM).

Polyplexes are formed when cationic polymers are mixed with DNA. Cationic polymers used to from polyplexes are of two general types: linear polymers such as polylysine and spermine; and the branched chain, spherical, or globular polycations such as polyethyleneimine and dendrimers. Lipopolyplexes are formed by the incorporation of polylysine into a lipoplex to form ternary complexes. DNA can be complexed with a natural biopolymer, e.g., gelatin or chitosan, functioning as a gene carrier to form nanospheres. Such biodegradable nanospheres have several advantages, including the coencapsulation of bioactive agents, e.g. nucleic acids and drugs, and the sustained release of the DNA. GelatinDNA or chitosan-DNA nanospheres are synthesized by mixing the DNA solution with an aqueous solution of gelatin or chitosan.

The effectiveness nonviral vectors used to deliver a ZPR1 nucleic acid for treating spinal muscular atrophy may be enhanced by conjugation to ligands that direct the vector either to a particular cell type or to a particular location within a cell. Antibodies and other site-specific proteins can be attached to a vector, e.g., on the surface of the vector or incorporated in the membrane. Following injection, these vectors bind efficiently and specifically to a target site. With respect to liposomes, ligands to a cell surface receptor can be incorporated into the surface of a liposome by covalently modifying the ligand with a lipid group and adding it during the formation of liposomes. The following classes of ligands can be incorporated into the nonviral DNA delivery complexes in order to make them more effective for gene delivery: (1) peptides, e.g., peptides having a specific cell surface receptor so that complexes will be targeted to specific cells bearing the receptor; (2) nuclear localization signals, e.g., to promote efficient entry of DNA into the nucleus; (3) pH-sensitive ligands, to encourage endosomal escape; (4) steric stabilizing agents, to prevent destabilization of the complexes after introduction into the biological milieu. Gene chemistry approaches, e.g. peptide nucleic acids, can be used to couple ligands to DNA to improve the in vivo bioavailability and expression of the DNA.

In plasmid-based, non-viral gene delivery systems it is often useful to link a polypeptide (e.g., an antibody), nucleic acid molecule, or other compound to the gene delivery plasmid such that the polypeptide, nucleic acid molecule or other compound remains associated with the plasmid following intracellular delivery in a manner that does not interfere with the transcriptional activity of the plasmid. This can be accomplished using an appropriate biotin-conjugated peptide nucleic acid (PNA) clamp. A sequence complementary to the biotin-conjugated PNA clamp is inserted into the gene delivery plasmid. The biotin-conjugated PNA will bind essentially irreversibly to the complementary sequence inserted into the plasmid. A polypeptide, nucleic acid molecule or other compound of interest can be conjugated to streptavidin. The streptavidin conjugate can bind to the biotin-PNA clamp bound to the plasmid. In this manner, a polypeptide, nucleic acid molecule or other compound can be bound to a gene delivery plasmid such that the polypeptide, nucleic acid molecule or other compound remains bound to the plasmid even within a cell. Importantly, the PNA clamp-binding site in the plasmid must be chosen so as not to interfere with a needed promoter/enhancer or coding region or otherwise disrupt the expression of the gene in the plasmid. An alternative approach employs a maleimide-conjugated PNA clamp. Polypeptides, nucleic acid molecules and other compounds containing a free thiol residue may be conjugated directly to the maleimide-PNA-DNA hybrid. As with the biotin-conjugated method, this conjugation does not disturb the transcriptional activity of the plasmid if the PNA-binding site is chosen to be in a region of the plasmid not essential for gene activity. Both of these approaches are described in detail by Zelphati et al. (2000, BioTechniques 28:304-315).

### EXAMPLES

### Example 1: Methods

Antibodies

Murine monoclonal antibodies to human ZPR1 (clones LG1, LG3, LG5, LG7, and LG18) were prepared using standard methods (Harlow and Lane, 1988, Antibodies: A Laboratory Manual, Cold Spring Harbor Press, Cold Spring Harbor, N.Y.). Purified antibodies to ZPR1 (e.g., LG1) were coupled to fluorescein isothiocyanate (FITC) using the Quick Tag FITC conjugation kit (Roche Molecular Biochemicals). Monoclonal antibodies to SMN were purchased from Transduction Labs (clone 8) and provided by Dr. G. Dreyfuss (clone 2B1; Liu and Dreyfuss, 1996, EMBO J. 15:3555-3565). The monoclonal antibodies to Sm (clone Y12) and the Flag epitope (clone M2) were purchased from NeoMarkers and Sigma, respectively. Sm is a protein that is a component of small nuclear ribonucleoprotein (e.g., see Brahms et al., 2000, J. Biol. Chem. 275:17122-17129).

Plasmids

Mammalian expression vectors for human ZPR1 (Galcheva-Gargova et al., 1998, Mol. Bio. Cell, 9:2963-2971) and SMN Liu et al., 1997, Cell 90:1013-1021) were constructed by sub-cloning cDNAs in the Eco R1/Xho I sites of pCDNA3 (Invitrogen). Bacterial expression of human ZPR1 was performed by sub-cloning a ZPR1 cDNA in the Eco R1/Xho I sites of pGEX-5x-2 (Amersham Pharmacia Biotech). Plasmids expressing full length and truncated Flag-tagged mouse ZPR1 have been described (Galcheva-Gargova et al., 1998, supra; Gangwani et al., 1998, J. Cell Biol. 143:1471-1484). Mutations were constructed by PCR using standard methods.

Cell culture

A431, COS7 and HeLa cells were maintained in Dulbecco's modified Eagle's medium (DMEM), 2 mM glutamine, 100 units/ml penicillin, 100 units/ml streptomycin, and 10% heat-inactivated fetal bovine serum (FBS). Wild type human fibroblasts (WI-38) were cultured in minimal Eagle's medium (MEM) with 10% FBS and 1 mM sodium pyruvate. Fibroblasts from human patients with Werdnig-Hoffman disease (SMA Type-I) were obtained from the Coriell Cell Repository (New Jersey, USA). Werdnig-Hoffman disease fibroblasts were Coriell Nos. GM03813, GM09677 and GM0232A. Other cell lines used were cells from the clinically unaffected parents of GM03813 (Coriell No. GM03814 (Mother) and GM03815 (Father)) were maintained in MEM with vitamins, 2× amino acids, and 20% FBS. Plasmid transfection assays were performed using Lipofectamine (Life Technologies Inc.).

Anti-Sense Oligonucleotides

Second generation optimized antisense oligonucleotides were purchased from Oligos Etc Inc. The anti-sense human ZPR1 oligonucleotide 5′-CATGGCCACCACGCGCAATT-3′ (SEQ ID NO:3) and two control oligonucleotides 5′-TTAACGCGCACCACCGGTAC-3′ (reverse sequence; SEQ ID NO:4), and 5′-CACGGCTACCTCGCACAAGT-3′ (scrambled sequence; SEQ ID NO:5) were examined. The oligonucleotides (0.4 nmoles) in 100 μl Opti-MEM (Life Technologies Inc.) were mixed with 100 μl Opti-MEM containing 6 μl Lipofectin (Life Technologies Inc.), diluted to 1 ml with MEM, and incubated (270 minutes) with sub-confluent HeLa cells in 35 mm dishes at 37° C. One milliliter of DMEM with 20% FBS was then added and the cells were incubated at 37° C. for 30 hours.

Immunoprecipitation Analysis

Cell extracts prepared using Triton lysis buffer (TLB: 20 mM Tris-HCl, pH 7.4, 137 mM NaCl, 2 mM EDTA, 25 mM β-glycerophosphate, 2 mM Na pyrophosphate, 1% Triton™ X-100, 10% glycerol, 1 mM PMSF, 1 mM Na orthovanadate, 10 μg/ml leupeptin, and 10 μg/ml aprotinin). Immunoblot analysis was performed by probing PVDF membranes with monoclonal antibodies to ZPR1 (LG1 and LG7), SMN (Transduction Laboratories) and Flag (M2; Sigma) in Tris-buffered saline containing 0.5% Tween-20 and 20% horse serum. Immunecomplexes were detected with HRP-conjugated sheep anti-mouse IgG (1:5000) secondary antibodies and enhanced chemiluminescence (Amersham Pharmacia Biotech). Immunoprecipitation assays were performed with the Flag antibody M2 pre-bound to protein G-Sepharose (Amersham Pharmacia Biotech) and a rabbit antibody to ZPR1 (Galcheva-Gargova et al., 1998, Science 272:1797-1802) pre-bound to 20 μl protein A-Sepharose (Amersham Pharmacia Biotech). The beads were incubated with cell lysate for two hours, washed three times, and boiled in Laemmli sample buffer prior to gel electrophoresis.

In Vitro Pre-mRNA Splicing

Synthesis of capped and [³²P] labeled pre-mRNA substrate was performed using the template pSP65-MINX linearized with Bam HI (Zillmann et al., 1988, Mol. Cell. Biol. 8:814-821) and the Riboprobe in vitro transcription system (Promega Corp). In vitro splicing reactions were performed using the Promega RNA splicing system. The effect of antibodies and proteins on in vitro splicing reactions was examined as described in Pellizzoni et al. (1998, Cell 95:615-624). Antibodies and GST fusion proteins were purified by column chromatography using protein A-Sepharose and glutathione-Sepharose (Amersham Pharmacia Biotech), respectively, and dialyzed extensively against buffer D (20 mM HEPES, pH 8.0, 20% glycerol (v/v), 0.2 mM EDTA, 0.1 M KCl, 0.5 mM DTT and 0.5 mM PMSF). In vitro splicing reactions were incubated at 30° C. for 60 minutes and treated with proteinase K (1.0 mg/ml) at 65° C. for 20 minutes. The RNA was extracted with phenol/chloroform, precipitated with ethanol, separated on a 12% polyacrylamide/urea denaturing gel, and examined by autoradiography.

Immunofluorescence Analysis.

To examine the localization of ZPR1 and SMN, cells were cultured on glass cover slips, rinsed with phosphate-buffered saline (PBS), and then rinsed briefly with chilled methanol. The cells were fixed at −20° C. in methanol for five minutes and with acetone for two minutes. The cover slips were blocked with 3% bovine serum albumin in PBS with 0.5% Tween-20 (PBS-T) for 30 minutes at 25° C. in Coplin jars and then incubated with primary antibodies for 1 hour at 25° C. Double labeling was carried out by sequential incubations (for 1 hour each) with anti-SMN (Clone 2B1; Liu and Dreyfuss, 1996, EMBO J. 15:3555-3565), secondary anti-mouse Texas-red antibody (Jackson ImmunoResearch) and then with FITC-conjugated LG1 (anti-ZPR1) at 25° C. The cells were rinsed once and washed with PBS-T (0.2% Tween-20), three times (for five minutes each) between each antibody incubation. The cover slips were mounted on slides using Vectashield with DAPI (Vector Laboratories) and examined by immunofluorescence microscopy using a conventional microscope Axioplan2 coupled with a MicroImager CCD camera (Carl Ziess). Three-dimensional images were recorded on an Olympus inverted microscope IX-70 fitted with a thinned back-illuminated cooled CCD Camera (Princeton Instruments) using a 100× objective (1.4 numerical aperture). Data voxel sizes were 120 nm×120 nm×200 nm with restoration on the same grid and 60 nm×60 nm×100 nm with subvoxel restoration on a 30 nm×30 nm×50 nm grid (Carrington et al., 1995, Science 268:1483-1487). The microscope point-spread function was calculated on a sub-pixel grid from an image of a 175 nm fluorescence bead (Carrington et al., supra). The restored images were visualized in three-dimensions using the volume visualization software DAVE (Lifshitz et al., 1994, In: Proceedings of the Biomedical Imaging Workshop. Institute for Electrical and Electronic Engineers, Computer Soc. Press, Los Angeles, pp. 166-175).

### Example 2: Interaction of SMN and ZPR1 in Transfection Assays

To discover the function of ZPR1, the interaction of ZPR1 polypeptide with other proteins was investigated. Several proteins were detected that co-precipitate with ZPR1 polypeptide from lysates of [³⁵S]methionine-labeled cells. In additional experiments, COS7 cells were transfected with plasmid vectors that express Flag-ZPR1, Flag-ZPR1ΔB, or SMN polypeptide. One of these co-precipitating proteins (40 kD) was identified as SMN. Cell extracts were prepared and ZPR1 polypeptide was immunoprecipitated with the Flag epitope tag antibody M2. The immunoprecipitates were examined by immunoblot analysis with an SMN antibody. Proteins in the cell lysates were examined by immunoblot analysis.

The deletion analyses performed using transfection assays demonstrated that the ZPR1 B domain is required for interaction of ZPR1 polypeptide with SMN polypeptide. These experiments indicated that the COOH-terminal region of ZPR1 (B domain) was required for interaction with SMN polypeptide and that the COOH-terminal region of SMN (corresponding to exon 7) was required for interaction with ZPR1 polypeptide. The reduced binding of SMN lacking exon 7 sequences is significant because the expression of SMNΔexon7 in the absence of full-length SMN polypeptide is associated with Werdnig-Hoffman syndrome (SMA Type-I). Together, these data indicate that SMN polypeptide can interact with ZPR1 polypeptide and that the COOH-terminal region of both proteins is required for optimal interaction. Mutations in the ZPR1 B-domain are thus candidates for causing spinal muscular atrophy or related disorders. Compounds that facilitate (e.g., stabilize) the interaction between the COOH-terminal regions of SMN and ZPR are especially likely to be useful for treating SMN.

### Example 3: In Vivo Interaction of SMN and ZPR1

The observation that SMN polypeptide and ZPR1 polypeptide interact in transfection assays indicates that these proteins may interact in vivo. To test this hypothesis, two methods were used to examine the interaction of endogenous ZPR1 polypeptide with SMN polypeptide.

First, the ability of ZPR1 polypeptide and SMN polypeptide to coprecipitate was investigated. In these experiments, A431 cells grown at 37° C. in medium containing 5% calf serum (control) were then incubated in serum-free medium for 24 hours (starved) and then treated (for 12 hours.) with 10% calf serum (serum). Extracts were prepared and ZPR1 was immunoprecipitated without antibody (beads) or with an antibody to ZPR1 bound to protein A-Sepharose. The amount of ZPR1 and SMN that coprecipitated was examined by immunoblot analysis using antibodies to ZPR1 (LG7) and SMN. These experiments showed that SMN co-immunoprecipitated with ZPR1. The extent of co-precipitation was reduced when the cells were serum-starved, but was induced following serum treatment. These data indicate that ZPR1 and SMN may form complexes in vivo.

In a second method of investigating whether SMN polypeptide and ZPR1 polypeptide interact in vivo, SMN and ZPR1 localization was examined using immunofluorescence analysis of starved and serum-stimulated cells. Note that SMN polypeptide is present in the cytoplasm and in the nucleus where it accumulates in the punctate structures known as gems. In these experiments, A431 cells were grown as described above and examined by conventional double-label immunofluorescence microscopy using monoclonal antibodies to ZPR1 polypeptide and SMN polypeptide. Co-localization of FITC-labeled ZPR1 and SMN indirectly stained with Texas-red conjugated antibody was detected by yellow fluorescence. DNA was stained with DAPI.

In these experiments, diffuse cytoplasmic staining was detected for both SMN polypeptide and ZPR1 polypeptide in serum-starved cells. In contrast, only a low level of SMN polypeptide and ZPR1 polypeptide was detected in the cytoplasm of serum-stimulated cells and both proteins were found to co-localize in punctate structures within the nucleus. These punctate structures correspond to nuclear gems, which are often found associated with coiled bodies and nucleoli. The co-localization of ZPR1 and SMN was observed in several cell types (fibroblasts, HeLa, A431, and COS7 cells) and also in experiments using different monoclonal antibodies to ZPR1 (clones LG1, LG3, and LG5) and SMN (clone 2B1) and clone 8 (Transduction Labs)). These data indicate that both SMN and ZPR1 exhibit mitogen-stimulated redistribution (and possibly recycling) from the cytoplasm to nuclear gems. The serum-induced redistribution to nuclear gems correlates with the serum-induced interaction of SMN with ZPR1.

Nuclear SMN is required for pre-mRNA splicing, most likely because it is needed for the regeneration of snRNPs (Pellizzoni et al., 1998, Cell, 95:615-624). To test whether ZPR1 might have a similar activity, an in vitro pre-mRNA splicing assay used (described by Pellizzoni et al., supra). In these experiments HeLa cell nuclear extract (50 μg) was incubated without (control) or with 2 μg of non-immune IgG, or with monoclonal antibodies to ZPR1 (clones LG1 and LG7), to SMN (Transduction Labs), and Sm proteins (clone Y12). In vitro splicing reactions were initiated by addition of pre-mRNA and the reactions were terminated after incubation for 60 minutes at 30° C. The pre-mRNA (zero minutes of incubation) and the reaction products were separated by gel electrophoresis and visualized by autoradiography. Control experiments demonstrated that antibodies to SMN and Sm proteins inhibited pre-mRNA splicing in vitro. Decreased pre-mRNA splicing was also detected in experiments using two different ZPR1 monoclonal antibodies (clones LG1 and LG7), but not in experiments using non-immune antibody. Thus, antibodies to ZPR1, like antibodies to SMN, inhibit pre-mRNA splicing in vitro.

The effects of recombinant GST or GST-ZPR1 on the in vitro splicing reaction was also examined. HeLa cell nuclear extracts were incubated without or with 0.04 mg/ml GST or GST-ZPR1. In vitro splicing reactions were initiated by addition of pre-mRNA and the reactions were terminated after incubation for 60 minutes at 30° C. No changes in splicing were observed. Similarly, recombinant SMN did not alter splicing, but, consistent with the Pellizzoni observations, inhibition was caused by a mutated SMN protein.

The effect of deletion of the B domain of ZPR1 (residues 298-459) was also examined. This deletion provides a putative dominant-negative ZPR1 protein with a COOH-terminal truncation that does not bind SMN (see Example 2). Genetic analysis has demonstrated that this mutated ZPR1 protein does not support viability in vivo (Galcheva-Gargova et al., 1998, Mol. Biol. Cell 9:2963-2971; Gangwani et al., 1998, J. Cell Biol. 143:1471-1484). To examine the effect of deletion of the B domain, HeLa cell nuclear extracts were incubated without or with 0.04 mg/ml GST or GST-ZPR1. In vitro splicing reactions were initiated by addition of pre-mRNA and the reactions were terminated after incubation for 60 minutes at 30° C. The effect of deletion of the B domain of ZPR1 (residues 298-459) was then examined.

It was discovered that that dominant-negative ZPR1 (ΔB) inhibits pre-mRNA splicing. Marked inhibition of pre-mRNA splicing in vitro was observed. The observation that both SMN and ZPR1 influence the same biochemical process, the splicing of pre-mRNA in vitro, provides support for the hypothesis that SMN and ZPR1 functionally interact in vivo. A dominant-negative ZPR1 mutation can also indicate the presence of SMA or a predisposition to SMA. Thus, detection of a dominant-negative mutation is useful for such diagnoses.

SMA Type-I is associated with mutation of the telomeric copy of the SMN gene (SMN1) which normally expresses the full-length SMN protein (Lefebvre et al., 1995, Cell, 80:155-165; Monani et al., Hum Mol. Genet. 1999, 8:1177-1183; Lorson et al., 1999, Proc. Natl. Acad Sci USA 96:6307-6311). In contrast, the centromeric copy of the SMN gene (SMN2) expresses primarily alternatively spliced forms of SMN lacking exon 7 (Monani et al., supra; Lorson et al., supra). Since the interaction of ZPR1 with SMN was markedly reduced by deletion of exon 7 sequences, the question was investigated of whether cells from SMA patient have a disrupted the interaction between ZPR1 and SMN. To test this, the interaction of SMN with ZPR1 in fibroblasts isolated from patients with SMA Type-I was examined. To perform these experiments, ZPR1 was immunoprecipitated from WT normal human diploid (SMN1+/+) fibroblasts (WI-38) and fibroblasts from patients with Werdnig-Hoffman disease (SMA Type-I) (SMN1−/−) (Coriell Nos. GM03813, GM09677, and GM0232A). Control experiments were performed with fibroblasts from the clinically unaffected parents (SMN1+/−) of GM03813 (mother (GM03814) and father (GM03815)). The presence of SMN in the ZPR1 immuneprecipitates was detected by immunoblot analysis as described above. The amount of ZPR1 and SMN in the cell lysates was also examined by immunoblot analysis.

It was found that SMN was not co-immunoprecipitated with ZPR1 in experiments using fibroblasts derived from three different SMA Type-I patients (SMN1−/−). In contrast, SMN was co-immunoprecipitated from normal human fibroblasts (SMN1+/+) and fibroblasts obtained from the parents (SMN1+/−) of one SMA Type-I patient (GM03813). Although co-immunoprecipitation was observed in experiments using cells from both parents, the extent of co-precipitation was greater for the mother (GM03814) than the father (GM03815). This defect in the father's cells correlates with a markedly reduced level of SMN expression compared with normal fibroblasts. Together, these data demonstrate that SMA Type-I is associated with defects in ZPR1/SMN complex formation in vivo.

The sub-cellular distribution of ZPR1 and SMN in SMA Type-I fibroblasts using immunofluorescence analysis was also examined. Fibroblasts from a patient (GM03813) with Werdnig-Hoffman disease (SMA Type-I) and his clinically unaffected parents (mother (GM03814) and father (GM03815)) were grown in medium supplemented with 20% FBS. The cells were processed for immunofluorecence by staining with monoclonal antibodies to SMN (2B1) and ZPR1 (LG1). The colocalization of FITC-ZPR1 and SMN indirectly stained with Texas Red was detected as yellow fluorescence. DNA was stained with DAPI. The images were obtained by conventional fluorescence microscopy.

A diffuse cytoplasmic distribution of ZPR1 and SMN was detected in these experiments. Punctate staining of nuclear gems by antibodies to ZPR1 and SMN was only detected in cells derived from the parents. A lower amount of nuclear gem staining was observed in the father's cells compared to the mother's cells, and correlates with reduced expression of SMN. The observation that SMA Type-I affects the nuclear gem localization of both SMN and ZPR1 suggests that the location of ZPR1 in gems is dependent on SMN.

These experiments illustrate methods that can be used to diagnose SMA. The immunocytochemical methods are also useful for determining whether compounds have an effect on ZPR1 localization.

### Example 4: ZPR1 and SMN are Required for Localization in Nuclear Gems

The mis-localization of ZPR1 in SMA Type-I cells indicates that the presence of SMN and ZPR1 in nuclear gems might require the interaction of these proteins. This possibility is consistent with the observation that serum stimulation induces both SMN/ZPR1 complex formation (Example 3). To further examine the hypothesis, the interdependence of ZPR1 and SMN on their localization in nuclear gems was examined.

To examine the role of ZPR1, the effect of ZPR1 depletion using anti-sense oligonucleotides was investigated. In these experiments, HeLa cells were transfected without (control) and with a ZPR1 antisense oligonucleotide. Control experiments were performed using cells transfected with an anti-sense oligonucleotide with the reversed ZPR1 sequence or with an oligonucleotide with a scrambled sequence as described in Example 1. The expression of ZPR1 and SMN in transfected cells was examined by immunoblot analysis of cell lysates. Transfection of HeLa cells with an anti-sense oligonucleotide decreased the expression of ZPR1 without changing the expression of SMN. Reverse sequence and scrambled sequence oligonucleotides did not cause marked changes in ZPR1 expression. Immunofluorescence analysis demonstrated that the ZPR1 anti-sense oligonucleotide disrupted the nuclear gem localization of both ZPR1 and SMN. In contrast, the reverse sequence oligonucleotide and the scrambled sequence oligonucleotide caused no change in SMN and ZPR1 localization in nuclear gems. Complementation analysis was used to further examine this phenomenon. In the complementation experiments, HeLa cells transfected without (control) and with an anti-sense ZPR1 oligonucleotide or a reversed sequence oligonucleotide were processed for immunofluorescence microscopy. Complementation experiments were performed by co-transfecting a ZPR1 expression vector (pCDNA3-hZPR1) together with the anti-sense oligonucleotide. The cells were stained with monoclonal antibodies to SMN (2B1) and ZPR1 (LG1) and colocalization was detected as described above using conventional fluorescence microscopy. The complementation experiments demonstrated that the disrupted localization of SMN and ZPR1 was restored by over-expression of recombinant ZPR1. These data indicate that ZPR1 is required for the localization of SMN in nuclear gems. They also show that overexpression of ZPR1 can be useful to correct mis-localization.

The observation that ZPR1 is mis-localized in SMA Type-I fibroblasts suggests that SMN may be required for the localization of ZPR1 in nuclear gems. To test this hypothesis, we performed complementation assays to examine whether ectopic expression of SMN in SMA Type-I cells is able to restore the localization of ZPR1 in nuclear gems. For these experiments, fibroblasts from a patient with Werdnig-Hoffman disease (SMA Type-I; GM03813) were transfected with an SMN expression vector (pCDNA3-SMN), incubated for 24 hours, and processed for immunofluorescence microscopy. The cells were stained with monoclonal antibodies to SMN (2B1) and ZPR1 (LG1). The colocalization of ZPR1 and SMN was detected as described above using conventional fluorescence microscopy. Immunofluorescence analysis demonstrated a diffuse cytoplasmic distribution of SMN and ZPR1 in SMA Type-I cells. In contrast, ectopic expression of SMN induced a punctate distribution of both SMN and ZPR1. The punctate pattern included nuclear structures, but additional punctate staining was observed in the cytoplasm, most likely as a consequence of SMN over-expression. These data indicate that SMN is required for the localization of ZPR1 in nuclear gems.

### Example 5: Interaction of ZPR1 and SMN in Nuclear Gems

Defects in either SMN or ZPR1 result in the mis-localization of both proteins and the failure to accumulate in nuclear gems. To examine the relationship between ZPR1 and SMN in nuclear gems, we used fluorescently labeled antibodies and three-dimensional digital microscopy with image restoration (deconvolution). In these experiments A431 cells in serum-free medium were processed for immunofluorescence analysis and were stained with DAPI and with monoclonal antibodies to SMN (red) and ZPR1 (green). Optical sections were collected and processed with 200 nm z-plane spacing and 120 nm per pixel (x, y) resolution. Images showing 50, 40, 30, 20, 15, and 10 planes of one representative cell were preserved. Control experiments using starved cells demonstrated that ZPR1 and SMN were present in the cytoplasm and that these proteins do not co-localize. Analysis of optical sections through the z-axis shows that serum starvation leads to the loss of nuclear gems and the redistribution of ZPR1 and SMN to separate compartments within the cytoplasm. In contrast, ZPR1 and SMN were found to co-localize in the nuclear gems of serum-treated cells. Optical sections of cell nuclei were collected and processed with 200 nm z-plane spacing and 120 nm per pixel (x, y) resolution. The average size of the ZPR1/SMN gems ranged between 0.6-1.2 μm and each nucleus contained 2-8 gems. Enlargement of this image indicated that the nuclear gems appear to contain overlapping staining of ZPR1 and SMN and that the staining patterns of ZPR1 and SMN are not identical. Greater image resolution was required to gain insight into the difference between the distribution of ZPR1 and SMN within a nuclear gem. We therefore restored images of nuclear gems within a 3×3 ×2.5 μm³ volume to sub-voxels of 30×30×50 nm (Carrington et al., supra). High resolution immunofluorescence images of nuclear gems within a single cell show similarities, but also significant differences. Three-dimensional images of these SMN/ZPR1 complexes indicated an interdigitated pattern with partial overlap between the staining by SMN and ZPR1 antibodies. However, the complexity and size of the structures differed between the nuclear gems. The difference in size and relative amounts of ZPR1 and SMN may reflect the dynamic state of gem assembly in living cells. Analysis of the three-dimensional images revealed that ZPR1 forms finger-like structures while SMN forms an oval-shaped cylinder with a central cavity. ZPR1 covers 75% of the SMN cylinder and caps one end. ZPR1 and SMN are therefore intimately associated within nuclear gems. This organization of ZPR1 and SMN may represent an essential structure that is required for the formation of complex multi-protein nuclear particles (gems).

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of determining the presence of or a predisposition for spinal muscular atrophy in a mammal, the method comprising,
(a) obtaining a biological sample from the mammal; and
(b) detecting one or more of:
(1) the amount of ZPR1 (zinc finger protein 1) RNA or ZPR1 polypeptide present,
(2) the level of ZPR1 protein activity per unit protein,
(3) the level of ZPR1 polypeptide interaction with survival of motor neuron protein (SMN) polypeptide, or
(4) ZPR1 polypeptide localization, relative to a control biological sample, wherein a difference in the amount of ZPR1 RNA or ZPR1 polypeptide, ZPR1 protein activity, the level of ZPR1 polypeptide interaction with SMN polypeptide, or ZPR1 polypeptide localization in the sample compared to the control indicates that the mammal has spinal muscular atrophy or has a genetic predisposition to spinal muscular atrophy.

2. The method of claim 1, wherein the mammal is a human.

3. The method of claim 1, wherein the amount of ZPR1 RNA or ZPR1 polypeptide, ZPR1 protein activity, or the level of ZPR1 polypeptide interaction with SMN polypeptide is decreased in the sample compared to the control.

4. The method of claim 1, wherein the biological sample includes cells containing nuclear gems, and wherein ZPR1 polypeptide localization to the nuclear gems is reduced in the sample compared to the control.

5. A method of determining the presence of or a predisposition to spinal muscular atrophy in a mammal, the method comprising,
(a) obtaining a biological sample from the mammal, and
(b) detecting a mutation in at least two zinc finger protein 1 (ZPR1) genes,
wherein mutations in two ZPR1 genes indicates that the mammal has spinal muscular atrophy or has a predisposition for spinal muscular atrophy.

6. The method of claim 5, wherein the mutation is in a ZPR1 B domain.

7. The method of claim 5, wherein the mammal is a human.

8. A method of determining the presence of or a predisposition for spinal muscular atrophy, or the spinal muscular atrophy carrier status of a mammal, the method comprising,
(a) obtaining a biological sample from the mammal, and
(b) detecting a mutation in a ZPR1 gene,
wherein the presence of a mutation indicates spinal muscular atrophy, a predisposition to spinal muscular atrophy, or spinal muscular atrophy carrier status.

9. The method of claim 8, wherein the mammal is a human.

10. The method of claim 8, wherein the ZPR1 gene encodes the amino acid sequence of SEQ ID NO:1.

11. The method of claim 8, wherein the mutation is in a ZPR1 B domain.

12. An isolated ZPR1 B domain polypeptide having the amino acid sequence of SEQ ID NO:2.

13. A method of using an effective amount of the isolated ZPR1 B domain of claim 12 or a fragment thereof to produce an antibody, the method comprising immunizing a mammal with the ZPR1 B domain or fragment thereof.

14. The method of claim 13, wherein the antibody is a polyclonal antibody.

15. The method of claim 13, wherein the antibody is a monoclonal antibody.

16. A purified antibody that specifically binds to a ZPR1 B domain.

17. An antibody made by the method of claim 13.

18. The antibody of claim 17, wherein the antibody is a monoclonal antibody.

19. A fusion polypeptide comprising a ZPR1 B domain fused to one or more non-ZPR1 polypeptides.

20. The fusion polypeptide of claim 19, wherein at least one non-ZPR1 polypeptide is a FLAG peptide.

21. A method of using the fusion polypeptide of claim 12 to produce an antibody, the method comprising immunizing a mammal with an effective amount of the fusion polypeptide.

22. An antibody made by immunizing a mammal with the fusion polypeptide of claim 19.

23. The antibody of claim 22, wherein the antibody specifically binds to a ZPR1 B domain.

24. A method of determining whether a compound alters the localization of a ZPR1 polypeptide, the method comprising
a) providing a test cell that expresses a ZPR1 polypeptide,
b) contacting the test cell with a candidate compound, and
c) detecting the localization of ZPR1 polypeptide in the test cell compared to a control cell not contacted with the candidate compound,
wherein a difference in the localization of ZPR1 polypeptide in the test cell indicates that the compound affects the localization of ZPR1 polypeptide.

25. The method of claim 24, wherein the compound affects the localization of ZPR1 polypeptide in nuclear gems.

26. A method of identifying a compound that modulates the interaction of a ZPR1 polypeptide and an SMN polypeptide, the method comprising
a) providing a test cell that expresses a ZPR1 polypeptide and an SMN polypeptide,
b) contacting the test cell with a candidate compound, and
c) detecting the interaction of the ZPR1 polypeptide and the SMN polypeptide compared to a control cell not contacted with the candidate compound,
wherein a difference in the interaction of the ZPR1 polypeptide and the SMN polypeptide relative to the control cell indicates that the compound modulates the interaction of ZPR1 polypeptide and SMN polypeptide.

27. The method of claim 26, wherein the interaction between the ZPR1 polypeptide and the SMN polypeptide is decreased.

28. The method of claim 26, wherein the interaction between the ZPR1 polypeptide and the SMN polypeptide is increased.

29. The method of claim 26, wherein the interaction is specific binding.

30. The method of claim 26, wherein the interaction is co-localization.
